(19) United States
(12) Patent Application Publication
(10) Pub. No.: US 2002/0164744 A1
(43) Pub. Date: Nov. 7, 2002

(54) Soluble, active recombinant human MAO-B

(76) Inventors: Martin R. Deibel JR., Kalamazoo, MI (US); Ronald D. Klein, Schoolcraft, MI (US); Anthony W. Yem, Kalamazoo, MI (US)

Correspondence Address: MARSHALL, GERSTEIN & BORUN, 6300 SEARS TOWER, 233 SOUTH WACKER, CHICAGO, IL, 60606-6357, US

(21) Appl. No.: 10/121,498

(22) Filed: Apr. 12, 2002

Related U.S. Application Data
(63) Continuation of application No. 09/368,117, filed on Aug. 4, 1999.

(51) Int. Cl. C12N 9/06; C07H 21/04; C12P 21/02; C12N 5/06

(52) U.S. Cl. 435/191; 435/69.1; 435/320.1; 435/325; 536/23.2

(57) ABSTRACT

Monoamine oxidase B is an integral polypeptide of the outer mitochondrial membrane. Purified, soluble, biologically active human MAO-B is obtained by recombinant methods in amounts useful for high volume screening.

### FIELD OF THE INVENTION

The invention relates to novel methods of preparing soluble, purified, biologically active recombinant human MAO-B.

### BACKGROUND OF THE INVENTION

Monoamine oxidases are higher eucaryote integral proteins of the outer mitochondrial membrane. These flavoenzymes catalyze the oxidative deamination of primary amines by a reaction between dioxygen and R—CH₂—NH₂ to form R—CHO, NH₃, and H₂O₂. Monoamine oxidases also act on secondary and tertiary amines and are important in the catabolism of neuroactive amines, e.g. epinephrine, norepinephrine, serotonin, and tyramine. Monoamine oxidases (MAO) are expressed in almost all tissues of vertebrates in two distinct forms, MAO-A and MAO-B. These two isoforms are distinguished by their difference in substrate preference and inhibitor specificity. MAO-A oxidizes vasoactive and neuroactive amines, such as serotonin, and is preferentially inactivated by the acetylenic inhibitor clorgyline. MAO-B, on the other hand, metabolizes xenobiotic amines and is preferentially inactivated by pargyline.

MAO-A and MAO-B, both of approximately 65 kDa molecular weight, are derived from two separate genes and have a 70% sequence identity on the amino acid level. Both genes have identical exon-intron organization, e.g., both genes have 15 exons and in exon 12 they possess a sequence coding for a cysteine residue that covalently binds flavin adenine dinucleotide (FAD). It is still unclear which features of their respective structures underlie the differences between the isoenzymes with regard to their kinetic properties. Numerous studies have suggested an involvement of these enzymes in the neuronal dysfunction of Alzheimers and Huntington's diseases as well as other neuropsychiatric diseases. Oreland, L., Monoamine Oxidase: Basic and Clinical Aspects, Yasuhara et al., Eds 219-247 (1993).

During the last three decades, many attempts have been made to purify these proteins from different natural sources such as liver tissue from pigs, [Oreland, L., Arch. Biochem. Biophys. 146:410-421 (1971)], rats, [Stadt, et al., Arch. Biochem. Biophys. 214:223-230 (1982)], beef, [Salach, J., Arch. Biochem. Biophys. 192:128-137 (1979)], and humans, [Dennick, et al., Biochem. J. 161:167-174 (1977)], as well as human platelets, [Szutowicz, et al., Biochem. Med. Metab. Biol. 36:1-7 (1986)], and placenta, [Weyler, et al., J. Biol. Chem. 260:13199-13207 (1985)]. The similarities of the physicochemical properties of MAO-A and MAO-B in the same tissues have made it difficult to separate the two isoforms from each other. Moreover, there have been great difficulties with the solubilization of the enzyme protein, e.g., the liberation of the enzyme from the outer mitochondrial membrane where it is anchored by its carboxy terminal. Although several hundred fold purification has been achieved by various techniques, solubility and contamination with the MAO-A isoform remain a problem. See Youdium, et al., Methods Enzymol. 142:617-627 (1987), Salach, et al., Methods Enzymol. 142:627-634 (1987).

The first reported cloning of human MAO-A and MAO-B was by Bach, et al., PNAS 85:4934-4938 (1988). The clone was from a human liver cDNA library and the authors were able to express MAO-B as a fusion protein in E. coli but did not purify the polypeptide or test for activity. The authors were only able to detect the presence of MAO-B with a specific monoclonal antibody, MAO B-1 C2, in an ELISA (enzyme linked immunosorbent assay) and by immunoblot analysis. Expression of human MAO-A and MAO-B was also reported in mammalian cells (HEK-293 cells) by Gottowik, et al., Eur J Biochem 230:934-942 (1995). However, expression levels of MAO-B were once again very low. Furthermore, the expressed MAO-B was never purified. Lu, et al., Protein Expression and Purification 7:315-322 (1996), reported expression of catalytically active MAO-B in a bacterial system. MAO-B was expressed in the E. coli strain BL21 and SDS-PAGE (polyacrylamide gel electrophoresis) analysis of cells after 3 hours of induction with isopropyl-β-D-thiogalactoside (IPTG) showed a prominent band with the expected apparent molecular weight of 65 kDa. The biologically active MAO-B was found in the membrane fraction but the major part of the expressed protein was present in the form of inclusion bodies. The inclusion bodies, which did not show any MAO activity, were insoluble in physiological buffers. Although the inclusion bodies could be solubilized under denaturing conditions, refolding experiments were unsuccessful.

Expression of MAO-A cDNA in S. cerevisiae has been reported by Weyler, et al., Biochem. Biophys. Res. Commun. 173:1205-1211 (1990).

Although expression of human recombinant MAO-B has been reported by Urban, et al., FEBS 286:142-146 (1991) and Grimsby, et al., Life Sciences 58:777-787 (1996), neither author was able to produce purified, biologically active MAO-B. Urban, et al. expressed human recombinant MAO-B in S. cerevisiae using a pYeDP1/8-2 expression vector. This expression system generated functional MAO-B, but the protein was localized mainly in yeast mitochondria. As a result, biologically active MAO-B could not be purified without a loss of activity. Grimsby, et al. expressed human recombinant MAO-B in S. cerevisiae using a pYES2 expression vector. Again, the expression levels of MAO-B were quite low and the active protein was localized in yeast mitochondria with the associated solubility problems described above.

Using a recombinant system for generation of human MAO-B has many advantages over isolation from natural sources. Labs which undertake the purification from natural sources are exposed to substantial risks of serious diseases such as HIV. Purification of human MAO-B requires kilograms of human liver or other tissue and the protein is still contaminated with the human MAO-A isoform. Currently, most labs working with MAO-B use the bovine form of the enzyme which can be obtained from natural sources and purified. However, human MAO-B is more desirable since it is the human enzyme which would be targeted in human disease studies and the binding properties or kinetic constants of the human and bovine forms of the enzyme may be different. Thus there is a need for high level expression of purified, soluble, biologically active recombinant human MAO-B.

### SUMMARY OF THE INVENTION

The present invention provides purified, soluble, biologically active human MAO-B polypeptides, advantageously in quantities useful for high volume screening. Preferably, the MAO-B polypeptide comprises the MAO-B amino acid sequence set out in SEQ ID NO: 12.

The invention also embraces polynucleotides encoding the polypeptides of the invention. Preferred polynucleotides include (a) the MAO-B coding DNA sequence set forth in SEQ ID NO: 11, (b) polynucleotides encoding the MAO-B amino acid sequence of SEQ ID NO: 12 and (c) polynucleotides that hybridize to (a) or (b) under stringent conditions and that encode polypeptides with MAO-B activity. A purified, soluble, biologically active human MAO-B polypeptide with a HIS tag is also contemplated.

In other aspects, the invention relates to a recombinant expression system for such polynucleotides encoding human MAO-B that is capable of producing purified, soluble, biologically active MAO-B, including vectors containing polynucleotides encoding MAO-B operatively linked to suitable expression control sequences, recombinant host cells which are transformed with such polynucleotides, and cultures of such host cells expressing the recombinant protein. Preferred expression control sequences include constitutive promoters, e.g., yeast ADH1 promoter; preferred host cells include S. cerevisiae and P. pastoids.

The invention further provides methods for producing a MAO-B polypeptide comprising the steps of: (a) growing host cells of the invention under conditions appropriate for expression of the MAO-B polypeptide and (b) isolating the MAO-B polypeptide from the host cell or its medium of growth.

The foregoing aspects and additional aspects may be apparent from the detailed description and examples which follow.

### DETAILED DESCRIPTION OF THE INVENTION

“Monoamine oxidase B (MAO-B)” as used herein refers to a polypeptide having the MAO-B amino acid sequence of SEQ ID NO: 12 or variants thereof which exhibit the spectrum of activity understood in the art for MAO-B. As described above, MAO-B is a flavoenzyme which catalyzes the oxidative deamination of endogenous and dietary amines. The spectrum of activity understood in the art includes but is not limited to the ability to oxidatively deaminate substrates such as dopamine and 1-methyl-4-(1-methyl-2-pyrryl)-1,2,3,6-tetrahydropyridine. As ionizable amino and carboxyl groups are present in the molecule, a particular polypeptide may be obtained as an acidic or basic salt, or in neutral form. Further, individual amino acid residues in the chain may be modified by oxidation, reduction, or other derivatization, and the polypeptide may be cleaved to obtain fragments which retain MAO-B activity. MAO-B containing solutions can be assayed for activity as described in Flaherty, et al., J Med Chem, 39:4756-4761 (1996).

Modifications to the amino acid sequence of MAO-B polypeptide of SEQ ID NO: 12 by deletion, addition, or alteration of amino acids can be made without destroying MAO-B activity. Such substitutions or other alterations result in variant polypeptides having an amino acid sequence which is substantially equivalent to MAO-B and fall within the definition of MAO-B, e.g. polypeptides with MAO-B activity that are 85%, 90%, 95%, 98% or 99% identical to the amino acid sequence of SEQ ID NO: 12, as determined by the best fit, Smith, et al., Advances in Applied Mathematics 2:482-489 (1981), and alignment, Needleman, et al., J. Mol. Biol. 48:443-453 (1970), methods.

Variant polypeptides include those wherein conservative substitutions have been introduced by modification of polynucleotides encoding polypeptides of the invention. Conservative substitutions are recognized in the art in relation to classification of amino acids according to their related physical properties and can be defined as set out in Table I (from WO 97/09433, page 10, published Mar. 13, 1997 (PCT/GB96/02197, filed 9/6/96).

TABLE I
Conservative Substitutions I

| | |
|---|---|
| SIDE CHAIN CHARACTERISTIC | AMINO ACID |

| | | |
|---|---|---|
| Aliphatic | Non-polar | G A P I L V |
| | Polar-uncharged | C S T M N Q |
| | Polar-charged | D E K R |
| Aromatic | | H F W Y |
| Other | | N Q D E |

Alternatively, conservative amino acids can be grouped as defined in Lehninger, Biochemistry, Second Edition; Worth Publishers, Inc. NY:N.Y. (1975), pp. 71-77. as set out in Table II.

TABLE II
Conservative Substitutions II

| SIDE CHAIN CHARACTERISTIC | AMINO ACID |
|---|---|
| Non-polar (hydrophobic) | |
| Aliphatic | A L I V P |
| Aromatic | F W |
| Sulfur-containing | M |
| Borderline | G |
| Uncharged-polar | |
| Hydroxyl | S T Y |
| Amides | N Q |
| Sulfhydryl | C |
| Borderline | G |
| Positively charged (basic) | K R H |
| Negatively charged (acidic) | D E |

“Stringent” as used herein refers to conditions that are commonly understood in the art as stringent. Stringent conditions can include highly stringent conditions (e.g., hybridization to filter-bound DNA in 0.5 M NaHPO₄, 7% sodium dodecyl sulfate (SDS), 1 mM EDTA at 65° C., and washing in 0.1× SSC/0.1% SDS at 68° C.), and moderately stringent conditions (e.g., washing in 0.2× SSC/0.1% SDS at 42° C.

“Purified” refers to a polynucleotide or a polypeptide of interest present in the substantial absence of other biological molecules, e.g., other polynucleotides of different sequences, polypeptides of different sequences, and the like. “Purified” human MAO-B as used herein is completely free of human MAO-A and preferably substantially free of other polypeptides with MAO enzymatic activity and substantially free of proteolytic activity as well. Purified human MAO-B includes immobilized metal affinity chromatography (IMAC) purified MAO-B as illustrated in Example 3 below. Purified human MAO-B preferably appears as a single band at about 58-60 kDa on SDS Page. Purified human MAO-B also preferably has a specific activity of ≧50 moles/min/mg when measured using 1-methyl-4-(1-methyl-2-pyrryl)-1,2,3,6-tetrahydropyridine as a substrate. In one embodiment, the polypeptide is purified such that it has a specific activity of 65 nmoles/min/mg, more preferably at least a specific activity of 80 nmols/min/mg.

“Isolated” as used herein refers to a polynucleotide or polypeptide that is found in a condition other than its native environment. The polynucleotide or polypeptide has been separated from other biological macromolecules normally present in its natural source. “Isolated further includes recombinant human MAO-B in the host cell supematant, and in purified preparations. In one embodiment, the isolated polynucleotide or polypeptide is substantially free of other polynucleotides or polypeptides. The terms “isolated” and “purified” do not encompass polynucleotides or polypeptides present in their natural source.

“Soluble” as used herein means a polypeptide that is capable of being dissolved in physiological buffers. Physiological buffers include, but are not limited to, acetate, bicarbonate, bis-tris propane, borate, citrate, dimethylmalonate, glycinamide, glycylglycine, imidazole, phosphate, succinate, and Tris together with any suitable buffer substance.

“Biologically active” as used herein refers to an MAO-B polypeptide that retains at least one biological enzymatic activity of the native polypeptide. Biological activity of MAO-B containing solutions can be measured as described by Flaherty, et al., J. Med. Chem., 39:4756-4761, using 1-methyl-4-(1-methyl-2-pyrryl)-1,2,3,6tetrahydropyridine as a substrate.

The invention relates to polynucleotides coding human MAO-B including DNA and RNA, including cDNA, genomic DNA and mRNA, as well as wholly or partially chemically synthesized DNA molecules.

The invention further relates to recombinant purified soluble, active MAO-B polypeptides, including variants thereof and methods for producing recombinant soluble, active MAO-B polypeptides. MAO-B polypeptides in recombinant form can be obtained in quantity, can be modified advantageously through regulation of the post-translational processing provided by the host, and can be intentionally modified at the genetic or polypeptide level to enhance desirable properties. Thus, the availability of MAO-B polypeptides in recombinant form provides both flexibility and certain quantitative advantages which make possible applications for use of the polypeptides in high volume screening.

Eukaryotic microbes such as filamentous fungi or yeast are suitable hosts for MAO-B encoding vectors. Saccharomyces cerevisiae, or common baker's yeast, is the most commonly used among lower eukaryotic host microorganisms. However, a number of other genera, species, and strains are commonly available and useful herein, such as Schizosaccharomyces pombe [Beach and Nurse, Nature, 290:140 (1981); EP 139,383 published May 2,1985)] Kluyveromyces hosts (U.S. Pat. No. 4,943,529) such as, e.g., K. lactis (Louvencourt et al., J. Bacteriol., 737 (1983)), K. fragilis, K. bulgalicus, K. thermotolerans, and K. marxianus, yarrowia [EP 402,226], Pichia pastors [EP 183,070; Sreekrishna et al., J. Basic Microbiol., 28:265-278 (1988)], Candida, Trichoderma reesia [EP 244,234], Neurospora crassa [Case et al., Proc. Natl. Acad. Sci. USA, 76:5259-5263 (1979)], and filamentous fungi such as, e.g, Neurospora, Penicillium, Tolypocladium [WO 91/00357 published Jan. 10, 1991], and Aspergillus hosts such as A. nidulans [Ballance et al., Biochem. Biophys. Res. Commun., 112:284-289 (1983)] Tilbum et al., Gene, 26:205-221 (1983); Yelton et al., Proc. Natl. Acad. Sci. USA, 81:1470-1474 (1984)] and A. niger [Kelly and Hynes, EMBO J., 4:475-479 (1985)].

Prokaryotic cells, such as bacterial cells can serve as host cells for the invention. Potentially suitable bacterial strains include Escherichia coli, Bacillus subtilit, Salmonella typhimunium, or any bacterial strain capable of expressing heterlogous proteins.

Furthermore, higher eukaryotic host cells are potentially suitable for use with the present invention. These include, but are not limited to, eukaryotic hosts such as HeLa cells, Cv-1 cells, COS cells, and Sf9 cells.

In general terms, the production of a recombinant soluble, active MAO-B involves the following:

First, a DNA encoding the mature polypeptide or a fusion of the MAO-B polypeptide to an additional sequence which does not destroy its activity or to an additional sequence cleavable under controlled conditions to give an active polypeptide, is obtained. This sequence should be in excisable and recoverable form. The excised or recovered coding sequence is then placed in operable linkage with suitable control sequences in a replicable expression vector. Suitable control sequences include both inducible and constitutive promoters. The vector is used to transform the host and the transformed host is cultured under favorable conditions to effect the production of recombinant MAO-B. The MAO-B can then be isolated and purified as described in Example 3 below. Purification of recombinant MAO-B may be accomplished in a series of steps, rather than in one large extraction and purification effort. Purification can be achieved in a variety of ways known in the art for protein purification, including but not limited to, Ni-NTA chromatography, size exclusion chromatography, ultracentrifugation or any combination thereof.

In a preferred embodiment, the DNA sequence of SEQ ID NO: 11 is placed in operative linkage with a constitutive promoter such as ADH1, although other constitutive or inducible promoters known in the art may be used. A HIS₆ tag with an ALA₂ spacer is added to the MAO-B open reading frame to aid purification. The S. cerevisiae strain, INVSc1, is transformed with the resulting vector and grown in appropriate medium. Any yeast strain known in the art may be used, including P. pastoris and S. pombe. The transformants are then selected and the recombinant MAO-B is isolated and purified.

### EXAMPLES

The present invention is illustrated by way of the following examples. Example 1 describes the plasmid constructions. Example 2 describes the transformation and growth of the yeast strain INVSc1. Example 3 describes the production fermentation process as well as purification of the recombinant human MAO-B. Example 4 describes a method for detecting HIS-tagged MAO-B and an assay to measure reaction velocity. Example 5 describes a confocal microscopy assay to determine whether full-length and truncated recombinant human MAO-B are localized to mitochondria. The foregoing specification and examples are intended to illustrate the present invention and are not intended to limit the scope of the invention as set out in the appended claims.

### Example 1

### Plasmid Construction

Oligonucleotide primers for the cloning and characterization of monoamine oxidase B were designed to the published nucleotide sequence as shown in SEQ ID NO: 1. Amplification of the MAO-B open reading frame (ORF) was accomplished using Stratagene Opti-Prime Kit, 10× buffer 6 (100 mM Tris-HCl pH 8.8,15 mM MgCl₂, 750 mM KCl) and Perfect Match 0.05 U per reaction. PCR was carried out in 30 cycles of 45 seconds at 95” C; 45 seconds at 50° C.; 2 minutes at 72° C.; followed by 10 minutes for primer extension at 72° C. The amplification was done with primers RDK 965 (SEQ ID NO: 2), a sense primer made to the amino terminus and RDK 972 (SEQ ID NO: 3), an antisense primer to the carboxyl terminus. Both primers included an EcoRI site. Internal sense primers RDK 966 (SEQ ID NO: 4), RDK 968 (SEQ ID NO: 5) and RDK 970 (SEQ ID NO: 6) and antisense primers RDK 967 (SEQ ID NO: 7), RDK 969 (SEQ ID NO: 8) and RDK 971 (SEQ ID NO: 9) were used to confirm the identify of the 1.5 kb MAO-B open reading frame. A HIS tag was added to the ORF using he antisense primer RDK 979 (SEQ ID NO: 10) contained an EcoRI site, termination codon, codons encoding HIS₆ and ALA₂ spacer and carboxy terminal amino acids.

A. pYES2-MAO-B (including UTRs)

Clone 1595912, obtained from INCYTE (Palo Alto, Calif.), (Genbank accession no. M69177) contained a cDNA encoding human monoamine oxidase B directionally cloned into the plasmid vector pINCY using EcoRl and NotI linkers. Clone 1595912 was digested to completion with EcoRI and NotI, the reaction mixture fractionated through an agarose gel and a 2.4 kb fragment purified. The intact MAO-B cDNA, including 5′ and 3′ untranslated regions (UTR) plus the ORF are included in this fragment. The plasmid vector, pYES2, containing the galactose inducible GALL promoter, was digested to completion with EcoRI and NotI, the reaction mixture was fractionated through an agarose gel and the linear plasmid was purified. The 2.4 kb fragment was ligated into the linearized pYES2 resulting in the plasmid pYES2-MAO-B. The orientation and structure of pYES2-MAO-B(R/N) was confirmed by PCR and nucleotide sequence analysis.

B. pYcDE8-MAO-B and pYES2-MAO-B (ORF only)

The MAO-B ORF missing the UTRs was amplified using PCR primers RDK 965 (SEQ ID NO: 2) and RDK 972 (SEQ ID NO: 3). The purified 2.4 kb EcoRI/NotI fragment was used as the substrate. The resulting fragment was cloned directly into the E. coli vector pCR2.1 resulting in the plasmid pCR2.1-MAO-B. The plasmid, pCR2.1-MAO-B was cleaved with EcoRI and the reaction mixture fractionated through an agarose gel. The 1.5 kb fragment was purified and ligated into the yeast shuttle vectors pYcDE8(University of Washington, Seattle, Wash.), containing the constitutive ADH1 promoter, previously described in Klein et al, Curr. Genet. 13:29-35 (1998) and pYES2, (Invitrogen, Carisbad, Calif.), containing the galactose inducible GALL promoter, (obtained from Invitrogen, Inc. (Acc. no IG1142)), which had been previously linearized with EcoRI. The resulting plasmids pYcDE8-MAO-B-4 and pYES2-MAO-B-8 were in the antisense orientation whereas the plasmids pYcDE8-MAO-B-1 and pYES2-MAO-B-11 were in the sense orientation as determined by restriction analysis. The DNA sequence of the plasmid inserts was confirmed using nucleotide sequence analysis.

C. pYcDE8-MAO-B-HIS₆ and pYES2-MAO-B-HIS₆

A HIS₆ tag with an ALA₂ spacer was added to the MAO-B ORF by a PCR based strategy. The MAO-B ORF was amplified using PCR primers RDK 965 (SEQ ID NO: 2) and RDK 979 (SEQ ID NO: 10). The purified 2.4 kb EcoRI/NotI fragment was used as the substrate. These primers engineered an Eco RI site at the N-terminus and a hexahistidine tag preceded by an Ala-Ala spacer, a stop codon and an EcoRI site at the C-terminus. The resulting PCR reaction product was ligated into pCR2.1 and the resulting plasmid, pCR2.1 MAO-B-HIS₆ was characterized by PCR. The MAO-B-HIS₆ insert was cut out using EcoRI and ligated into the EcoRI site of the yeast shuttle vectors pYcDE8 and pYES2. MAO-B-HIS₆ was characterized by restriction mapping and nucleotide sequence analysis. The MAO-B insert consists of a 1.5 kb region of the gene beginning at nucleotide 78 and ending at nucleotide 1637 as shown in SEQ ID NO: 11.

D. C-terminal deletion constructs of MAOB-HIS₆

PCR primers were designed to amplify nucleotides 61-1571(Met1-Leu498); 61-1487(Met1-Val470); and 61-1331(Met1-Val418) from a MAO-B sequence amplified from the original MAO-B clone. An EcoRI site was engineered into the N-terminal end directly before the start codon. The C-terminal primers of each incorporated an Ala-Ala spacer, followed by a hexa-hisitdine tag, a stop codon and finally an EcoRI site. After EcoRI digestion and purification of the PCR products, the MAO-B inserts containing the HIS₆ tags were ligated into the yeast expression vector pYcDE8. The direction of the inserts was determined by restriction analysis and their sequence confirmed by DNA sequence analysis.

### Example 2

Transformation and Growth of Yeast Strain INVSc1

The yeast strain INVSc1 (α, his3-Δ1, leu2, trp1-289, ura3-52) was obtained from Invitrogen Corporation (Carlsbad, Calif.) and used in all studies requiring a S. cerevisiae host. Complete medium YEPD (2% glucose) was used for routine growth of INVSc1 as described by Adams, et al, Methods in Yeast Genetics, Cold Spring Harbor Laboratory Press (1997). YEPG (2% galactose) was used for GAL1ₚ induction and YEPR containing 2% raffinose, a non repressing and non inducing carbon source was used to prepare overnight cultures for galactose induction. Yeast transformants were selected and maintained on SCM-ura (pYES2) or SCM-trp (pYcDE8) containing dropout base media(DOB) (BIO 101 Inc., Vista CA). For galactose induction studies requiring selective medium DOB-galactose and DOB-raffinose were used instead of DOB.

The pYES2 plasmids containing MAO-B in both the sense and antisense orientations, in addition to vector alone were transformed into the yeast strain INVSc1 and plated on yeast SC-ura/glucose. Individual ura+transformants were then transferred into 10 ml of SC-ura/raffinose and placed on a rotary shaker overnight at 30° C. The nonreducing and nonrepressing sugar raffinose was used to avoid the possibility of glucose repression or galactose induction of the GAL1 promoter. 5 ml aliquots of the overnight culture were then used to inoculate 100 ml of YEPGAL or 100 ml of YEPRAF incubated for 6 hours and overnight, respectively, at 30° C. in a G-24 shaker and harvested by centrifugation. The cell pellet from YEPGAL was stored at −80° C. The cell pellet from the YEPRAF was washed once with YEPGAL and then resuspended in 100 ml YEPGAL and the cells incubated for 6 hours as described above. Additional studies were conducted using SC DOB-galactose and DOB-raffinose as described about but with incubation times ranging from 6 to 18 hours.

The pYcDE8 plasmids containing MAO-B in both sense and antisense orientations, in addition to the vector alone were transformed into yeast strain INVSc1 and plated on yeast SC-trp/glucose. Additionally, pYcDE8 plasmids containing the C-terminal deletion constructs were also transformed into yeast strain INVSc1 and plated on yeast SC-trp/glucose. Individual trp⁺ transformants were transferred into 10 ml of YEPD and placed on a rotary shaker overnight at 30° C. The 10 ml overnight was then used to inoculate 250 ml of YEPD, which was incubated for 24 hours before being placed overnight in a G-24 shaker and harvested by centrifugation. The cell pellets were then stored at −80° C.

### Example 3

### Production Fermentation and Purification of Recombinant Human MAO-B

0.1 ml of glycerol stock was inoculated into 100 ml volumes of filter sterilized DOB minus tryptophan medium contained in 500 ml wide mouth fermentation flasks. The inoculated flasks were incubated at 30° C. for 48 hours with agitation at 200 rpm. A secondary seed fermentation was inoculated at a 10% rate using the 48 hour primary seed culture and fermented using the described protocol with agitation at 250 rpm for 24 hours. The production fermentation was carried out in YEPD medium sterilized by autoclaving and was inoculated using the 24 hours secondary seed culture at rates ranging between 6 and 10%. The inoculated 100 ml volumes of YEPD contained in 500 ml wide mouth shake flasks were incubated at 30° C. with agitation at 250 rpm and harvested by centrifugation at 24 hours post inoculation. The production fermentations were carried out in 12 L volumes using 120 shake flasks per run. The wet weight cell yield was usually ca. 22 g/L of fermentation.

About 60 g (wet weight) of frozen (−70° C.) cell pellets derived from a 3 liter fermentation of yeast were thawed and resuspended in 250 ml of deionized water. The mixture was centrifuged at 3000 rpm for five minutes at 4° C. The cell pellet was resuspended in 50 mM Tris HCl, pH 7.5, 10 mM MgCl₂, 1 M Sorbitol, 30 mM DTT (zymolyase buffer) and incubated for 30 minutes at room temperature on a rocking mixer. The cells were collected by centrifugation as described above. This pellet was resuspended in the zymolyase buffer without the added reducing reagent. 100 mg of lyticase (Sigma Chem. Co., from Anthrobacter luteus, crude 1000 U/mg solid) were added and the suspension was incubated on an orbital shaker (200 rpm) overnight (approx. sixteen hours) at 18° C. The spheroplast pellet which resulted was washed three times with 100 ml of ice cold zymolyase buffer (redistributed into the suspension by gentle stirring with a spatula). The washed spheroplasts were last resuspended in 250 ml of lysis buffer (10 mM Tris HCl, pH 7.5, 50 mM NaCl, 1 mM EDTA, and 0.5% Triton X-100®. Protease inhibitors can also be added if desired. The suspension was then homogenized (18 strokes) in a Dounce homogenizer (40 ml capacity, plunger A, tight fitting). The yeast cell extract was collected by centrifugation (3200 rpm×5 min, Sorvall RT6000D, swinging bucket) and the pellet discarded. The extract was purified on an IMAC column as follows. Extract was mixed with 25 ml of packed Ni-NTA resin (Qiagen) equilibrated in the lysis buffer. This suspension was mixed on a rocker for approximately two hours at 8° C. to facilitate the affinity interaction between the Ni-NTA resin and the HIS tag on MAO-B. The matrix was washed thoroughly with lysis buffer, and then was re-equilibrated in lysis buffer in the absence of the detergent, Triton-X-100®. HIS-tagged MAO-B was eluted by rinsing the column with 50 ml of a 400 mM imidazole solution prepared in lysis buffer. The eluted MAO-B was effectively concentrated to 1-2 ml by ultrafiltration (YM-100 filter, Amicon). The concentrated MAO-B was stored in 50% glycerol at −20° C.

Additional washing steps were introduced to the protocol described above to limit the amount of non-specific protein binding to the IMAC column. After the equilibrated IMAC column was loaded with the yeast extract containing recombinant MAO-B, it was washed with 25-30 ml of 2 M NaCl in 50 mM Tris HCl, pH 8.0. The column was then washed with 50 mM Tris buffer to remove the excess NaCl before the elution step (400 mM imidazole in 50 mM Tris HCl, pH 8.0) as described above. Concentration steps relying on the use of stirred cells were avoided since lower molecular weight fragments of MAO-B were observed after such procedures. The concentration step using an Amicon stirred cell was replaced by a Centriprep-30, a device that does not require stirring and allows for protein to be concentrated away from the filtration membrane. This approach effectively lowered protein aggregation when compared to other centrifugal concentration devices where concentration of solute occurs at the membrane filter surface.

To further resolve MAO-B from other protein contaminants and breakdown products of MAO-B, the concentrated sample was loaded onto a Superose-6 (10 mm×30 cm—Pharmacia Biotech) size exclusion column that had been equilibrated in 1× PBS at a flow rate of 0.5 ml/min. Table III below shows yields of recombinant MAO-B from 60 g (wet weight) cell paste after each of the purification steps described above.

TABLE III
Yields of Recombinant Human MAO-B

| | AMOUNT | | |
|---|---|---|---|
| | OF MATERIAL | SPECIFIC | |
| | PRODUCED | ACTIVITY | Km FOR |
| | FROM APPROX. | (nmoles/ | SUBSTRATE |
| FRACTION | 60 g OF YEAST | min/mg) | (μM) |
| IMAC eluted/ | Approx. 3 ml @ 25 | 8.7 | 71.9 |
| concentrate | mg/ml total protein | | |
| Post-Superose 6 | Approx. 1-2 ml @ | 80.6 | 55.4 |
| chromatography/ | 0.5-1.0 mg/ml | | |
| concentrate | rhMAO-B protein | | |

Fractions were then analyzed by SDS polyacrylamide gel electrophoresis and pooled according to the purity of the fraction obtained.

### Example 4

### Detection of HIS tagged MAO-B and Activity Measurement

For detection of HIS tagged MAO-B, the polypeptide mixture was fractionated using SDS-polyacrylamide gel electrophoresis. The mixture was then transferred and immobilized on PVDF membranes (Millipore). Western blots were completed using a semi-dry electroblotter with a constant current set at 125 mA/gel (7/9 cm). Blots were visualized by either staining with Coomassie blue R250 (0.2% w/v) in 50% ethanol, 5% acetic acid followed by a destaining step using 50% ethanol, or alternatively, were processed for immunostaining.

For immunostaining, the blots were first blocked in 2% nonfat dried milk in 1× phosphate buffered saline for five minutes, washed in 20 mM Tris buffered saline (TBS), exposed to anti-HIS tag IgG (1:500 dilution) (Santa Cruz Biotechnology, Inc., Santa Cruz, Calif.) and incubated for one hour. The blots were washed in 0.5% Tween-20 in TBS for ten minutes followed by five minutes in TBS and then exposed to secondary antibody, anti-rabbit IgG (Fc) alkaline phosphatase conjugate (obtained from Promega Corp., Madison, Wis.), used at a 1:1000 dilution. The location of MAO-B on blots was identified using an alkaline phosphatase NBT/BCIP substrate system (BioRad Laboratories, Inc.). Following development, blots were air dried prior to storage.

The MAO-B containing solutions, including fractions isolated from chromatography columns, were assayed to measure reaction velocity. The assays were carried out in a final volume of 200 μl in 50 mM sodium phosphate, 10 mM NaCl, pH 7.5, with 0.2 mM substrate (1-methyl-4-(1-methyl-2-pyrryl)-1,2,3,6-tetrahydropyridine) as described in Flaherty, et al., J. Med. Chem., 39:4756-4761 (1996) hereby incorporated by reference. Reaction velocity was followed at an absorbence of 420 nm at 37° C. in a SpectraMax 250 96-well plate reader. Specific activity measurements were made using a Shimadzu UV-2101PC spectrophotometer equipped with a Shimadzu temperature controller (TCC-controller) set to 37° C. Protein concentrations were determined using the Bio-Rad Protein Assay kit (Bio-Rad Laboratories, Hercules, Calif.).

Analysis of samples containing purified wild-typeMAO-B was carried out using 1-methyl-4-(1-methyl-2-pyrryl)-1,2,3,6-tetrahydropyridine as the substrate. The effect of protein concentration on activity was measured. The reaction for each protein concentration was shown to have a linear time course at 37° C. for the duration of the assay (10-30 minutes). The plot of MAO-B concentration vs. activity was a straight line. Variation of the substrate at a constant enzyme concentration was used to determine the Kₘ for the substrate. The Michaelis curve revealed a typical saturation profile and the Lineweaver Burke plot was a straight line (K, calculated as 71.9 μM). Again, the reaction progress at each concentration of substrate was followed as straight lines with coefficients of correlation greater than 0.99.

The activity of samples containing the purified mutant MAO-B described in Example 1 above was also analyzed using 1-methyl-4-(1-methyl-2-pyrryl)-1,2,3,6-tetrahydropyridine as the substrate. Activity was highest for wild-type MAO-B followed by the MAO-B mutant Met1-Leu498 which had approximately 10% of wild-type activity. The two remaining C-terminal deletion mutants, Met1-Val470 and Met1-Val418, had about the same activities, less than 0.1% of wild-type MAO-B activity. This confirms that deletion or truncation of the C-terminus of human MAO-B causes loss of enzymatic activity.

### Example 5

### Mitochondrial Localization in COS cells

A cell based method was used to validate the mitochondrial localization of recombinant human full length MAO-B having a HIS tag, as well as to determine if a mutant form (22 C terminal amino acids deleted) of human MAO-B (also with a HIS tag) was localized in the mitochondria. DNA coding for the full length and truncated C-terminal HIS tagged MAO-B proteins, as described above, was ligated into the EcoRI sites of the mammalian expression vectors pcDNA3.1(Invitrogen) and pEGFP-C2 (Clontech). COS cells were transfected to produce transient expression of both proteins in each vector and the resulting transfectants were examined using confocal microscopy. The COS cells containing the pcDNA3.1/MAO-B (truncated and full-length) were permeabilized and examined by using immunofluorescence with a rabbit anti-HIS tag IgG (Santa Cruz). The antibody was purified by Protein A Sepharose, and then FITC tagged using a kit available from Sigma (Fluorotag FITC Conjugation kit). The COS cells transiently expressing the pEGFP-C2/MAO-B (truncated and full-length) were examined directly as live cells by confocal microscopy. The results of both experiments showed that the full-length, HIS tagged MAO-B protein bound to the mitochondria of the COS cells whereas the truncated form was found to be located in the cytoplasm or in intracellular vesicles such as lysosomes or protesomes.

We claim:

1. A purified, soluble, biologically active human MAO-B polypeptide comprising the MAO-B amino acid sequence set forth in SEQ ID NO: 12.

2. The purified, soluble, biologically active human MAO-B polypeptide of claim 1, further comprising a HIS tag.

3. The purified, soluble, biologically active human MAO-B polypeptide of claim 1, wherein said polypeptide has a specific activity of from about 40 nmoles/min/mg to about 90 nmoles/min/mg when measured by absorbance using 1-methyl-4-(1-methyl-2-pyrryl)-1,2,3,6-tetrahydropyridine as a substrate.

4. The MAO-B polypeptide of claim 3, wherein said polypeptide has a specific activity of at least 65 nmoles/min/mg.

5. The MAO-B polypeptide of claim 3, wherein said polypeptide has a specific activity of at least 80 nmoles/min/mg.

6. A purified, soluble, biologically active human MAO-B polypeptide encoded by the nucleotide sequence selected from the group consisting of a) the MAO-B coding nucleotide sequence set forth in SEQ ID NO: 11, b) a nucleotide sequence that encodes the MAO-B amino acid sequence of SEQ ID NO: 12, and c) a nucleotide sequence that hybridizes to a) or b) under stringent conditions.

7. A purified, soluble, biologically active human MAO-B polypeptide comprising an amino acid sequence with 90% or greater identity to the amino acid sequence of the polypeptide of claim 1.

8. A method for producing a purified, soluble, biologically active human MAO-B polypeptide comprising:
a) growing a host cell transformed with DNA encoding human MAO-B polypeptide under conditions permitting expression of said polypeptide; and
e) isolating soluble, biologically active MAO-B polypeptide from said host cell or its medium of growth.

9. The method of claim 8 wherein said DNA encodes the human MAO-B sequence of SEQ ID NO: 12.

10. The method of claim 8 wherein said DNA comprises the MAO-B coding nucleotide sequence of SEQ ID NO: 1 or 11.

11. The method of claim 8 wherein the host cell is transformed by a nucleotide sequence selected from the group consisting of (a) the MAO-B coding nucleotide sequence set forth in SEQ ID NO: 11, (b) a nucleotide sequence that encodes the MAO-B amino acid sequence of SEQ ID NO: 12, and (c) a nucleotide sequence that hybridizes to (a) or (b) under stringent conditions.

12. The method of claim 8 wherein the host cell is transformed with an expression vector comprising DNA encoding human MAO-B polypeptide.

13. The method of claim 12, wherein the expression vector comprises a constitutive promoter.

14. The method of claim 13, wherein the constitutive promoter is ADH1.

15. The method of claim 8 wherein the host cell is yeast.

16. The method of claim 15 wherein the host cell is S. cerevisiae.

17. The method of claim 15 wherein the host cell is P. pastoris.


SEQUENCE LISTING (12 sequences)

SEQ ID NO: 1 - DNA, 2491 bp, Homo sapiens
ctggcaggcaggactgggatcgaggcccagaaaacggagcagcgggcaccagggaggcctggaacggggcgagcgccatgagcaacaaatgcgacgtggtcgtggtggggggcggcatctcaggtatggcagcagccaaacttctgcatgactctggactgaatgtggttgttctggaagcccgggaccgtgtgggaggcaggacttacactcttaggaaccaaaaggttaaatatgtggaccttggaggatcctatgttggaccaacccagaatcgtatcttgagattagccaaggagctaggattggagacctacaaagtgaatgaggttgagcgtctgatccaccatgtaaagggcaaatcataccccttcagggggccattcccacctgtatggaatccaattacctacttagatcataacaacttttggaggacaatggatgacatggggcgagagattccgagtgatgccccatggaaggctccccttgcagaagagtgggacaacatgacaatgaaggagctactggacaagctctgctggactgaatctgcaaagcagcttgccactctctttgtgaacctgtgtgtcactgcagagacccatgaggtctctgctctctggttcctgtggtatgtgaagcagtgtggaggcacaacaagaatcatctcgacaacaaatggaggacaggagaggaaatttgtgggcggatctggtcaagtgagtgagcggataatggacctccttggagaccgagtgaagctggagaggcctgtgatctacattgaccagacaagagaaaatgtccttgtggagaccctaaaccatgagatgtatgaggctaaatatgtgattagtgctattcctcctactctgggcatgaagattcacttcaatccccctctgccaatgatgagaaaccagatgatcactcgtgtgcctttgggttcagtcatcaagtgtatagtttattataaagagcctttctggaggaaaaaggattactgtggaaccatgattattgatggagaagaagctccagttgcctacacgttggatgataccaaacctgaaggcaactatgctgccataatgggatttatcctggcccacaaagccagaaaactggcacgtcttaccaaagaggaaaggttgaagaaactttgtgaactctatgccaaggttctgggttccctagaagctctggagccagtgcattatgaagaaaagaactggtgtgaggagcagtactctgggggctgctacacaacttatttcccccctgggatcctgactcaatatggaagggttctacgccagccagtggacaggatttactttgcaggcaccgagactgccacacactggagcggctacatggagggggctgtagaggccggggagagagcagcccgagagatcctgcatgccatggggaagattccagaggatgaaatctggcagtcagaaccagagtctgtggatgtccctgcacagcccatcaccaccacctttttggagagacatttgccctccgtgccaggcctgctcaggctgattggattgaccaccatcttttcagcaacggctcttggcttcctggcccacaaaagggggctacttgtgagagtctaaagagagagggtgtctgtaatcacactctcttcttactgtatttgggatatgagtttggggaaagagttgcaagtaaagttccatgaagacaaatagtgtggagtgaggcgggggagcatgaagataaatccaactctgactgtaaaatacaatggtatctctttctccgttgtggcccctgcttagtgtcccttacctggcttagcgttctgtttcaccagtttccaagtttattgccctcaaatctttagaatagttaaattggcttgtttaaggttcttgctgccccacaacacaccttgcccatgcacaggatgaattttttcctaccattatggctttgtgcttgttcttcctcttacctgtatagcctcacttccctagttctttgcattcgtccttaggtactgtattgttacagctgaaagacagtaaagaccatttagtcctcaccttctgttttagagttgagcaaactgaagcccacagaggtggaacttaattacctaagagccacaataagccactggtatctgggggactagaacacaaataattgcttttcccacctctttggatgttttccccaattatcctccttcactccctgtcatagttaccgatggtgtcccgttgtgtgggtttactctgtgctaagttgtcttacacttctcaaatgctactcagtatatagccttaactcttactgttttgtgcggtgtgtctccagctgattttaacttttttgatggtagaaattttatctcttcttccttttgtatcctccattgtatcttcatacaaaggacagtacacacttgggtaattaaaaataaaagttgattgaccat

SEQ ID NO: 2 - DNA, 24 bp, Homo sapiens
ggcctggaattcggggcgagcgcc

SEQ ID NO: 3 - DNA, 29 bp, Homo sapiens
gaagagagtgtgaattcagacaccctctc

SEQ ID NO: 4 - DNA, 20 bp, Homo sapiens
gagagattcccgagtgatgc

SEQ ID NO: 5 - DNA, 22 bp, Homo sapiens
ctcgtgtgcctttgggttcagt

SEQ ID NO: 6 - DNA, 19 bp, Homo sapiens
ggagggggctgtagaggcc

SEQ ID NO: 7 - DNA, 20 bp, Homo sapiens
atggggcatcactcggaatc

SEQ ID NO: 8 - DNA, 19 bp, Homo sapiens
ctgaacccaaaggcacacg

SEQ ID NO: 9 - DNA, 21 bp, Homo sapiens
gcctctacagccccctccatg

SEQ ID NO: 10 - DNA, 65 bp, Homo sapiens
cccggatcctagtgatggtgatggtgatgggctgcgactctcacaagtagccccttttgtgggcc

SEQ ID NO: 11 - DNA, 1619 bp, Homo sapiens
  CDS (25)..(1608)
ggcctggaattcggggcgagcgccatgagcaacaaatgcgacgtggtcgtggtggggggcggcatctcaggtatggcagcagccaaacttctgcatgactctggactgaatgtggttgttctggaagcccgggaccgtgtgggaggcaggacttacactcttaggaaccaaaaggttaaatatgtggaccttggaggatcctatgttggaccaacccagaatcgtatcttgagattagccaaggagctaggattggagacctacaaagtgaatgaggttgagcgtctgatccaccatgtaaagggcaaatcataccccttcagggggccattcccacctgtatggaatccaattacctacttagatcataacaacttttggaggacaatggatgacatggggcgagagattccgagtgatgccccatggaaggctccccttgcagaagagtgggacaacatgacaatgaaggagctactggacaagctctgctggactgaatctgcaaagcagcttgccactctctttgtgaacctgtgtgtcactgcagagacccatgaggtctctgctctctggttcctgtggtatgtgaagcagtgtggaggcacaacaagaatcatctcgacaacaaatggaggacaggagaggaaatttgtgggcggatctggtcaagtgagtgagcggataatggacctccttggagaccgagtgaagctggagaggcctgtgatctacattgaccagacaagagaaaatgtccttgtggagaccctaaaccatgagatgtatgaggctaaatatgtgattagtgctattcctcctactctgggcatgaagattcacttcaatccccctctgccaatgatgagaaaccagatgatcactcgtgtgcctttgggttcagtcatcaagtgtatagtttattataaagagcctttctggaggaaaaaggattactgtggaaccatgattattgatggagaagaagctccagttgcctacacgttggatgataccaaacctgaaggcaactatgctgccataatgggatttatcctggcccacaaagccagaaaactggcacgtcttaccaaagaggaaaggttgaagaaactttgtgaactctatgccaaggttctgggttccctagaagctctggagccagtgcattatgaagaaaagaactggtgtgaggagcagtactctgggggctgctacacaacttatttcccccctgggatcctgactcaatatggaagggttctacgccagccagtggacaggatttactttgcaggcaccgagactgccacacactggagcggctacatggagggggctgtagaggccggggagagagcagcccgagagatcctgcatgccatggggaagattccagaggatgaaatctggcagtcagaaccagagtctgtggatgtccctgcacagcccatcaccaccacctttttggagagacatttgccttccgtgccaggcctgctcaggctgattggattgaccaccatcttttcagcaacggctcttggcttcctggcccacaaaagggggctacttgtgagagtcgcagcccatcaccatcaccatcactaggatccggg

SEQ ID NO: 12 - protein, 528 aa, Homo sapiens
MSNKCDVVVVGGGISGMAAAKLLHDSGLNVVVLEARDRVGGRTYTLRNQKVKYVDLGGSYVGPTQNRILRLAKELGLETYKVNEVERLIHHVKGKSYPFRGPFPPVWNPITYLDHNNFWRTMDDMGREIPSDAPWKAPLAEEWDNMTMKELLDKLCWTESAKQLATLFVNLCVTAETHEVSALWFLWYVKQCGGTTRIISTTNGGQERKFVGGSGQVSERIMDLLGDRVKLERPVIYIDQTRENVLVETLNHEMYEAKYVISAIPPTLGMKIHFNPPLPMMRNQMITRVPLGSVIKCIVYYKEPFWRKKDYCGTMIIDGEEAPVAYTLDDTKPEGNYAAIMGFILAHKARKLARLTKEERLKKLCELYAKVLGSLEALEPVHYEEKNWCEEQYSGGCYTTYFPPGILTQYGRVLRQPVDRIYFAGTETATHWSGYMEGAVEAGERAAREILHAMGKIPEDEIWQSEPESVDVPAQPITTTFLERHLPSVPGLLRLIGLTTIFSATALGFLAHKRGLLVRVAAHHHHHH